(19) United States
(12) Patent Application Publication
(10) Pub. No.: US 2009/0326281 A1
(43) Pub. Date: Dec. 31, 2009

(54) Catalytic system and process for direct synthesis of dimethyl ether from synthesis gas

(75) Inventors: Lucia Gorenstin Appel, Laranjeiras (BR); Andrea Maria Duarte De Farias, Vila da Penha (BR); Angela Maria Lavogade Esteves, Meier (BR); Marco Andre Fraga, Laranjeiras (BR); Flavia de Souza Ramos Barbosa, Tijuca (BR); Luiz Eduardo Pizzaro Borges, Ipanema (BR); Jose Luiz Fontes Monteiro, Jardim Guanabara (BR); Jhonny Oswaldo Huertas Flores, Copacabana (BR); Maria Isabel Pais Da Silva, Humaita (BR); Eduardo Falabella Sousa Aguiar, Gavea Rio de Janeiro (BR)

Correspondence Address: NIXON & VANDERHYE, PC, 901 NORTH GLEBE ROAD, 11TH FLOOR, ARLINGTON, VA, 22203, US

(73) Assignees: PETROLEO BRASILIERO S.A. - PETROBRAS, Rio de Janeiro (BR); INT - INSTITUTO NACIONAL de TECNOLOGIA, Rio de Janeiro (BR)

(21) Appl. No.: 12/458,004

(22) Filed: Jun. 29, 2009

(30) Foreign Application Priority Data
Jun. 30, 2008 (BR) PI 0803764-7

(51) Int. Cl. C07C 41/01; B01J 29/04

(52) U.S. Cl. 568/671; 502/60; 502/74

(57) ABSTRACT

A mixed-bed catalytic system and its activation for direct synthesis of dimethyl ether from synthesis gas are described, comprising a catalyst for methanol synthesis and the zeolite ferrierite in its acid form as the methanol dehydrating component, the two being mixed physically in the form of powder of defined granulometry or as pellets. Another object of the present invention is a process for production of the acid form of the zeolite ferrierite. Another object of the present invention is a process for direct synthesis of dimethyl ether from a synthesis gas, using the catalytic system of the present invention.

### FIELD OF THE INVENTION

The present invention relates to catalysts for direct synthesis of compounds from synthesis gas, more particularly for direct synthesis of dimethyl ether, and more specifically a catalytic system resulting from the physical mixing of a catalyst for methanol synthesis and a zeolite as dehydrating component.

### BACKGROUND OF THE INVENTION

Dimethyl ether, also known by the abbreviation DME, is appearing among companies and research centres in the more developed countries, as a promising energy alternative, replacing the petroleum derivatives, notably for Diesel and LPG.

One of the great advantages of DME is its flexibility with respect to the raw material. In fact, it can be obtained from coal, from petroleum residues, from natural gas or biomass residues.

This fuel has various advantages in relation to protection of the environment, where the main factor is the non-generation of particulates when employed in diesel engines.

Traditionally DME is produced on the basis of dehydration of methanol, employing catalysts with acidic characteristics.

On the other hand, methanol is produced by the hydrogenation of carbon monoxide.

Recently, attention has been directed towards the direct synthesis of dimethyl ether from a synthesis gas, using a catalytic system that combines a catalyst for methanol synthesis and a catalyst for dehydration of said alcohol.

It was confirmed on the basis of theoretical and experimental studies that both the stage of methanol-synthesis and the stage of methanol dehydration could be conducted simultaneously on one and the same catalytic system, also known as a hybrid catalyst. To this set of reactions we must, moreover, add the reaction of displacement of water gas that would also occur simultaneously.

Direct synthesis makes it possible to overcome the thermodynamic limitations of methanol synthesis.

In fact, in the direct synthesis of dimethyl ether, the methanol is constantly withdrawn and dehydrated, which increases the conversion of carbon monoxide, displacing the equilibrium value of conversion to very high levels.

The majority of the patent documents relating to the direct synthesis of dimethyl ether from a synthesis gas have many similarities.

Generally a synthesis gas is used with H₂/CO ratio between 1 and 2, in a process that operates in a temperature range between 240° C. and 300° C., with pressure in the range from 3000 kPa to 6000 kPa, with space velocities in the range from 500 h⁻¹ to 5000 h⁻¹.

These patents employ hybrid catalysts composed of a catalyst for methanol synthesis, which generally contains the following elements: Cu/Zn, Zn/Al, Zn/Cr, Cu/Zn/Al, Cu/Zn/Cr, Cu/Zn/Co or Cu/Cr/Fe, and a catalyst for dehydration of methanol based on aluminas or zeolites. In the majority of the patent documents, the hybrid catalyst is formed by physical mixing of the two components.

Among the methanol synthesis catalysts, the one displaying the best results was CuO/ZnO/Al₂O₃, a catalyst usually employed on an industrial scale.

The dehydration of methanol to obtain dimethyl ether takes place on the acid sites of a porous material and, in the majority of the patent documents and scientific articles, gamma-alumina and the zeolites HZSM-5 and HY are cited as dehydrating components (Huang, Applied Catalyst A: General, 167, 1998, 23 and Li, Appl. Catal. A 147, 1996, 23). Some Chinese patent documents also cite the following zeolites: H-faujasite, mordenite and HY (1Ch-CN1087033).

### RELATED TECHNOLOGY

Based on studies of reaction mechanism, Schiffino et al. (J. Phys. Chem., 97, 1993, 6425) demonstrated that the dehydration of methanol takes place on the acid sites.

According to Takeguchi et al. (Applied Catalysis A: General 192, 2000, 201-209) the active centres for the dehydration of methanol would be the Brønsted acid sites and the Lewis acid-base pair.

Shen (Thermochimica Acta 434, 2005, 22-26) found that catalysts with strong Brønsted acid sites displayed high activity in terms of dehydration.

Conversely, Kim (Applied Catalysis A: General 264, 2004, 37-41) and Appel (Catalysis Today 101, 2005, 39-44) demonstrated that the rate of dehydration of methanol depends on the acid strength of the dehydrating components.

U.S. Pat. No. 4,375,424 (Slaugh), inserted here as reference, presents a catalyst and a process for the production of dimethyl ether from a synthesis gas, in which the catalyst is composed of copper and zinc supported on gamma-alumina with a surface area of about 150 m²/g and 500 m²/g, calcined in a temperature range from about 400° C. to 900° C. and reduced at a temperature of about 100° C. to 275° C. and where said catalyst has a sodium content of less than 700 ppm.

U.S. Pat. No. 3,894,102 (Chang et al.), inserted here as reference, shows conversion of synthesis gas to gasoline, in which the synthesis gas is contacted with a mixture of a hydrogenation catalyst and an acid dehydration catalyst, to produce dimethyl ether in a first stage. Then this substance must be brought in contact with a crystalline aluminosilicate so as to convert it to high-octane gasoline.

U.S. Pat. No. 4,520,216 (Skov et al.), inserted here as reference, shows that synthetic hydrocarbons, especially high-octane gasoline are prepared by means of a catalytic reaction of a synthesis gas containing hydrogen and oxides of carbon in two stages. In a first stage, the synthesis gas is converted to an intermediate containing methanol and/or dimethyl ether in the following conditions: 1000 kPa to 8000 kPa and 200° C. to 300° C. The catalysts that can be used for the synthesis of methanol are oxides of chromium, aluminum and/or copper, and zinc; and, for the synthesis of dimethyl ether, certain zeolites. In the second stage, the intermediate from the first stage is converted completely, using inlet temperatures of 300° C. to 340° C. Heat is supplied throughout the reactor to make it possible to reach outlet temperatures of 410° C. to 440° C.; the difference between the inlet temperature and the outlet temperature must be at least 30° C. greater than the temperature increase due to the reaction. As catalyst in the second stage, it is possible to use some conventional catalysts for conversion of methanol and/or dimethyl ether to hydrocarbons, especially synthetic zeolites. The product obtained in the second stage is cooled and separated into two streams: a mixture of condensed hydrocarbons and recycle gases. The latter are recycled and combined with the fresh feed of synthesis gas. A low rate of deactivation of the catalyst used in the second stage and a mixture of hydrocarbons of high quality are observed.

Chinese patent CN 1085824 (Guangyu et al.), inserted here as reference, describes a catalyst and a process for production of dimethyl ether with synthesis gas as raw material. The catalyst is formed from a type of catalyst for industrial synthesis of methanol and alumina that was modified with oxide of boron, titanium or phosphorus. The catalyst has a simple process for preparation, displays high catalytic activity, good selectivity for dimethyl ether and is stable during the reaction. The technology involves, in addition to the direct preparation of dimethyl ether from synthesis gas, also procedures for separation. The method uses ethanol or water as extractant and dimethyl ether with purity greater than 99% can be obtained directly at low pressure.

Japanese patent JP 63254188 A2 (Masaaki et al.), inserted here as reference, teaches the production of hydrocarbons from a synthesis gas, obtaining a liquefied fraction with high octane index, bringing a synthesis gas in contact with a catalyst for methanol synthesis and a dehydrating agent to form dimethyl ether and CO₂, separating CO₂ from the uncondensed gas by means of a membrane and bringing the purified gas in contact with a zeolite.

In fact, the acidity is the most relevant property of the dehydrating component.

Furthermore, it was verified by Appel et al. (Catalysis Today 101, 2005, 39-44) that the greater the acidity, the higher the rate of formation of DME. Conversely, it was observed that, for systems of high acidity, this rate is a function of the rate of formation of methanol.

It then proves very desirable, for carrying out the synthesis of DME adequately, to have an acid material with a large number of strong Brønsted acid sites.

### SUMMARY OF THE INVENTION

The use of porous acidic materials, such as zeolites, has given good results in the direct synthesis of dimethyl ether, principally by presenting a high acid strength and a large number of Brønsted sites.

One object of the present invention is a mixed-bed catalytic system and activation thereof for direct synthesis of dimethyl ether from synthesis gas, which comprises a catalyst for methanol synthesis and the zeolite ferrierite in its acid form as the methanol dehydrating component, the two being mixed physically in the form of powder of defined granulometry or as pellets.

The catalytic system obtained is selective for dimethyl ether and does not exhibit formation of unwanted products such as methane and hydrocarbons, for example.

Another object of the present invention is a process for production of the acid form of the zeolite ferrierite.

The zeolite H-ferrierite, the acid form of the zeolite ferrierite, has a silica/alumina ratio equal to 10 and has a content by weight of potassium and sodium of the order of 5.2% and 0.9%, respectively.

Another object of the present invention is a process for direct synthesis of dimethyl ether from a synthesis gas, using the catalytic system of the present invention.

### BRIEF DESCRIPTION OF THE DRAWINGS

FIG. 1 is a graphical representation illustrating the spectrum in the infrared region of pyridine adsorbed at 25° C. on zeolite H-ferrierite after exposure to vacuum.

FIG. 2 is a graphical representation of the profile of programmed-temperature desorption of ammonia on zeolite H-ferrierite.

FIG. 3 is a graphical representation of the conversion of the CO present in synthesis gas to dimethyl ether at different flow rates of the feed gas.

FIG. 4 is a graphical representation of the molar selectivity for CO₂ at different flow rates of the feed gas.

FIG. 5 is a graphical representation of the molar selectivity for methanol at different flow rates of feed gas.

FIG. 6 is a graphical representation of the molar selectivity for dimethyl ether at different flow rates of feed gas.

FIG. 7 is a graphical representation of stability in the conversion of the synthesis gas to dimethyl ether.

### DETAILED DESCRIPTION OF THE INVENTION

The present invention relates to a mixed-bed catalytic system for direct synthesis of dimethyl ether from synthesis gas, which comprises a catalyst for methanol synthesis and the zeolite H-ferrierite as the methanol dehydrating component, a process for production of the acid form of the zeolite ferrierite, and a process for direct synthesis of dimethyl ether from a synthesis gas, using the catalytic system of the present invention.

The catalytic system of the present invention comprises a catalyst for methanol synthesis and a zeolite ferrierite in its acid form.

The catalyst for methanol synthesis has a composition that can be selected from a mixture of copper oxide and zinc oxide, and it can also be composed only of copper oxide, zinc oxide and aluminum oxide. Other cations can also be added, for example: Zr, Cr, Ga, Pd, Pt, or other metals.

The catalyst for methanol synthesis can be prepared by co-precipitation from a mixture of a solution of nitrates of the metals of interest with a solution of calcium carbonate. The precipitate obtained is then calcined.

Alternatively, the catalyst for methanol synthesis can be selected from commercial catalysts for this purpose.

The use of porous acidic-materials, such as zeolites, has given good results in the direct synthesis of dimethyl ether, and the performance depends on the nature and concentration of acid sites, as already mentioned.

The zeolites are structures formed by a three-dimensional system of tetrahedra of aluminum (trivalent) and silicon (tetravalent), which are coordinated tetrahedrally with oxygen atoms. These tetrahedra are joined together by means of oxygen atoms that they have in common.

In this situation, each oxygen atom possesses, as close neighbours, two atoms of Al or two atoms of Si or even one atom of Al and one of Si. This last option causes a charge imbalance, because Al has lower valency and binding of a proton becomes necessary to produce a stable structure. The Brønsted acid site then arises.

The zeolite ferrierite proves to be a good dehydrating component due, principally, to the high concentration of Brønsted acid sites and to the high acid strength.

Ferrierite is a zeolite that belongs to the mordenite group and has two systems of channels. One has an elliptical section with dimensions of 4.2×5.4 Å and a cross-sectional area of approximately 18 Å. The second system of channels is formed from eight-membered rings with diameters of 3.5×4.8 Å. These channels are responsible for the properties of ferrierite and contain water and sodium and/or potassium ions to compensate the negative charge of the structure of the TO₄ tetrahedra (Datka, Applied Catalyst A: General 6414, 2003, 1-7 and Wichterlová, Microporous and Mesoporous Materials 24, 1998, 223-233).

The process for production of the acid form of the zeolite ferrierite, with silica/alumina ratio in the range of values between 60-5, takes place basically by ion exchange of the sodium and potassium ions of the ferrierite by NH₄⁺ ions using a solution of salts that can be selected from: ammonium nitrate, ammonium chloride and ammonium acetate, and which comprises the following steps:
bring in contact, in a reflux system, a mass of zeolite ferrierite with a solution of ammonium salt with a concentration greater than 1 mol/L, stirring continuously, at a temperature of 90° C., for a time that varies in the range from 1.5 to 3 hours;
wash the zeolite paste with deionized water corresponding to four times the final volume of the mixture, after the exchange;
repeat the preceding stages of exchange and washing;
dry the acidic zeolite at a temperature in the range from 80° C. to 120° C.;
calcine the acidic zeolite at a temperature in the range from 300° C. to 700° C. for a period of time in the range from 2 to 6 hours, at a heating rate in the range from 1° C./min to 10° C./min for removal of NH₄⁺ ions.

Preferably, the salt for ion exchange is ammonium nitrate; the solution of ammonium salt has a preferred concentration in the range from 1.5 mol/L to 1.7 mol/L; the stirring time is in the range from 2 to 2.5 hours; the drying temperature is preferably in the range from 90° C. to 100° C.; the preferred calcination temperature is in the range from 400° C. to 500° C.; the preferred calcination time is in the range from 4 to 5 hours; and the preferred heating rate is between 3° C./min and 8° C./min.

After the processing described above, the zeolite ferrierite has Brønsted acid sites and high acid strength, the main requirement for dehydration of the methanol that has formed.

Now moving on to presentation and explanation of the diagrams that form an integral part of the present specification, FIG. 1 shows a spectrum in the infrared region of the zeolite H-ferrierite after adsorption of pyridine at 25° C. and exposure to vacuum at 25° C. (A), 150° C. (B) and 250° C. (C). Bands of high intensity corresponding to pyridine coordinated with Lewis and Brønsted acid sites were observed. At 1444 cm⁻¹ we identified a band corresponding to the Lewis acid sites (Wichterlová, Microporous and Mesoporous Materials, 1998, 24, 223-233) and, at 1488 cm⁻¹, we identified a band corresponding to the Brønsted acid sites (pyridinium ions and binding by a hydrogen bridge) plus Lewis acid sites.

Furthermore, a very intense band was also observed at 1545 cm⁻¹, corresponding to the Brønsted sites (pyridinium ion), and another two bands were found at 1620 cm⁻¹ and 1635 cm⁻¹, identified as Lewis and Brønsted acid sites.

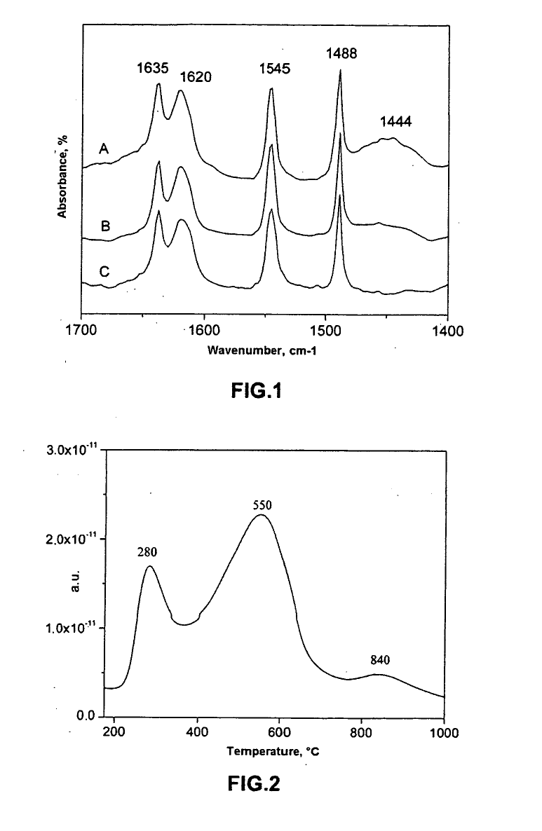

FIG. 2 shows the result of the programmed-temperature desorption of ammonia on zeolite H-ferrierite, where the presence of three desorption peaks is clearly observed at 280° C., 550° C. and 840° C., which can be identified as weak acid sites, strong acid sites and very strong acid sites, respectively. The overall acidity of zeolite H-ferrierite calculated on the basis of the programmed-temperature desorption of ammonia (DTP-NH₃) was 1608 μmol NH₃/g of sample, and 71% of this acidity would correspond to the strong acid sites, i.e., 1134 μmol NH₃/g.

The catalytic system for direct synthesis of dimethyl ether from a synthesis gas, one of the objects of the present invention, is prepared by the physical mixing of the catalyst for methanol synthesis and of the dehydration catalyst obtained as described above, both of them in the form of powder or pellets, where the molar ratio between the catalyst for methanol synthesis and the dehydration catalyst is in the range of values between 1 and 10.

Preferably, the ratio of the catalyst for methanol synthesis to the dehydration catalyst is in the range of values between 3 and 7.

Activation of the catalytic system is carried out with a reducing atmosphere of hydrogen in a gas mixture of H₂/He with molar concentration in the range from 3% to 10% of H₂, with a heating rate in the range from 1° C./min to 10° C./min for a period of time in the range from 40 to 80 min, and a reduction temperature in the range from 150° C. to 350° C.

The preferred values for the activation of the catalytic system are molar concentration in the range from 4% to 6% of H₂, at a heating rate in the range from 3° C. to 8° C., and up to a reduction temperature in the range from 200° C. to 300° C., for a period of time in the range from 50 min to 70 min.

For better understanding and assessment of the invention, we present the results of some laboratory experiments, which are purely for illustration, without limiting the invention.

### EXAMPLE 1

### Preparation of a Catalytic System for Direct Synthesis of Dimethyl Ether (Mixed Bed)

5 g of zeolite ferrierite (Toyo Soda Manufacturing Co., batch N° HZS-720 KOA) was weighed and put in a flask containing 75 mL of a solution of ammonium nitrate with a concentration of 1.5 mol/L and heated to 90° C., for a period of 2 hours with a reflux system to prevent evaporation of the solvent.

After this period of time, the zeolite was separated from the solution by centrifugation and washed with 1 L of deionized water. After washing, the zeolite paste obtained in the first exchange was again put in a flask containing the same volume of solution of ammonium nitrate of the same concentration and the reflux system was used, heating at 90° C. for a further period of 2 hours.

The zeolite was separated by centrifugation and washed with 1 L of deionized water. Then the zeolite was dried in a stove at 90° C. for a period of 12 hours.

This material was macerated and sieved to obtain a particle granulometry with size of 60 mesh. The zeolite was calcined in a nitrogen atmosphere (50 mL/min) at a temperature of 400° C. for a period of 4 hours, at a heating rate of 5° C./min.

Finally, approximately 0.05 g of the zeolite H-ferrierite obtained and 0.2 g of commercial catalyst for methanol synthesis were weighed. Both samples were pelletized before being assessed. Pellets with average size of 7 mm in diameter and with thickness of 3.3 mm, approximately, were used for the catalytic tests.

The process for direct synthesis of dimethyl ether comprises:
forming the catalytic system by the physical mixing of the catalyst for methanol synthesis with the dehydration catalyst zeolite H-ferrierite;
reducing the catalytic system with H₂/He gas mixture;
on completion of reduction, starting the feed of the synthesis gas according to the following parameters: the volume ratio H₂/CO of reaction is in the range of values between 0.5 and 5; the reaction temperature varies in the range of values between 200° C. and 300° C.; the reaction pressure varies in the range of values between 1000 kPa and 10000 kPa; and the space velocity of reaction is in the range of values from 2 h⁻¹ to 100 h⁻¹.

### EXAMPLE 2

### Synthesis of Dimethyl Ether

The process of synthesis of dimethyl ether from synthesis gas was carried out in a continuous unit comprising a Berty reactor and a Varian CP-3800 chromatograph coupled in line, equipped with two detectors: a thermal conductivity detector (TCD) and a flame ionization detector (FID).

The flow rate of the feed gas (1:1 mixture of H₂/CO) is controlled by a mass flow meter.

The volume of the reactor is 50 mL.

The Berty reactor is a reactor with internal recycling without temperature gradient, equipped with:
a fixed cylindrical basket that holds the catalyst;
an internal stirrer, which remains above the basket and promotes the motion of the gas in a downward direction along the reactor wall and returns, from below upwards, to enter the catalyst bed;
a device for temperature measurement and control;
a pressure gauge;
a thermostatic bath for cooling the connection between the reactor and the stirrer; and
a temperature-controlled electric stove.

The Varian chromatograph, which is coupled to the Berty reactor, is equipped with a chromatographic column (Varian), with H₂ as carrier gas. The programming of column temperature was 35° C. for 2 minutes, followed by a heating ramp in a range of values from 5° C./min up to 150° C./min.

The line connecting the reactor to the chromatograph has a micrometric valve that controls the pressure and is maintained at a temperature around 90° C., preventing the condensation of products.

The catalyst was placed in the basket of the reactor and sealed. Next, the stove was installed in the reactor and the stirrer was switched on. Catalyst reduction was carried out using H₂/He gas mixture (5% H₂) at a flow rate of 30 mL/min. Reduction was carried out at 250° C. for one hour, heating at a rate of 5° C./min.

On completion of reduction, feed of the synthesis gas was started. Both the temperature and the pressure were adjusted to the operating conditions of 250° C. and 5066 kPa.

The reaction products were analyzed in the chromatograph by regular injections (every half hour) executed by an automatic injection valve.

The first injection began 10 minutes after the start of passage of the gases. The next injections were at half-hourly intervals. Reaction was continued for 250 minutes.

The catalyst proved to be active and selective for the direct synthesis of dimethyl ether.

The results for the conversion at different flow rates of feed gas were found to be inversely proportional to the increase in said flow rate (10 mL/min (A), 18 mL/min (B) and 24 mL/min (C)), as could be foreseen.

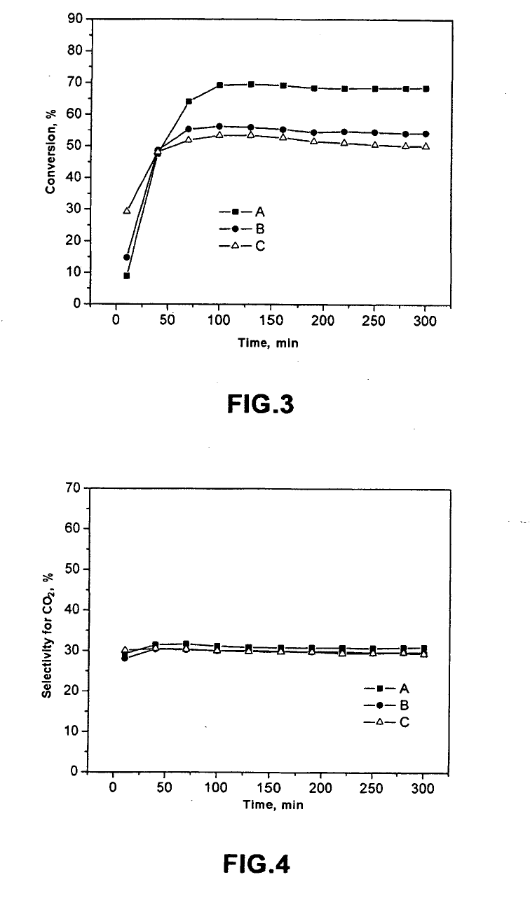

Conversion reached values of up to 70%, as can be followed graphically in FIG. 3.

Selectivity for CO₂ remained around 30% for 10, 18 and 24 mL/min, as can be seen and followed in FIG. 4.

Very little methanol was observed, the values observed being in the range from 2.6% to 3.7%, as can be followed graphically in FIG. 5.

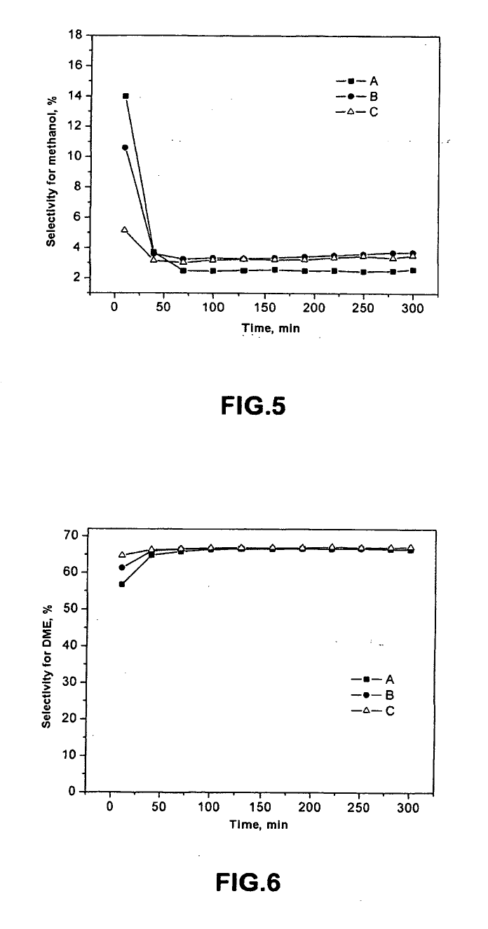

The molar selectivity in terms of dimethyl ether was 67% for flow rates of 18 mL/min (B), 24 mL/min (C) and 10 mL/min (A), as can be seen in FIG. 6.

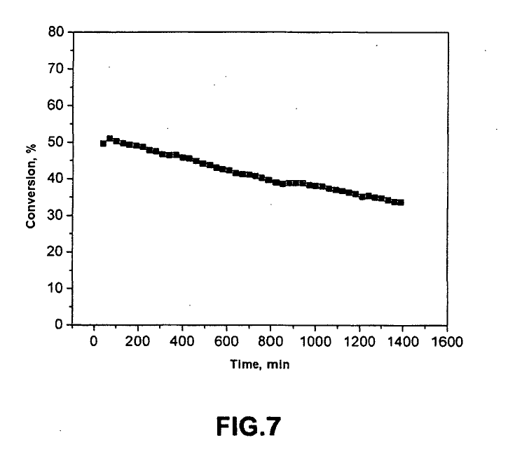

The catalyst showed a drop in conversion after 24 hours of reaction, as shown graphically in FIG. 7.

Although the present invention has been described in its preferred embodiment and with a representative example, the main concept that guides the present invention, which is a mixed-bed catalytic system for direct synthesis of dimethyl ether from synthesis gas, comprising a catalyst for methanol synthesis and the zeolite. H-ferrierite as the methanol dehydrating component, a process for obtaining the completely acid form of the zeolite ferrierite and a process for direct synthesis of dimethyl ether from a synthesis gas, using the catalytic system of the present invention, is preserved with respect to its innovative character, where a person skilled in the art might envisage and carry out variations, modifications, changes, adaptations and the like that are conceivable and compatible with the operating means in question, though without departing from the scope and spirit of the present invention, which are represented by the claims given hereunder.

1. Mixed-bed catalytic system for direct synthesis of dimethyl ether from synthesis gas, which comprises a catalyst for methanol synthesis and a methanol dehydration catalyst, characterized in that the methanol dehydration catalyst is a zeolite ferrierite in its acid form, the two being mixed physically in a form that can be selected from powder of defined granulometry and pellets, and where the ratio of the catalyst for methanol synthesis to the methanol dehydration catalyst is in the range of values between 1 and 10.

2. Catalytic system according to claim 1, characterized in that the ratio of the catalyst for methanol synthesis to the dehydration catalyst is in the range of values between 3 and 7.

3. Catalytic system according to claim 1, characterized in that the catalytic system is activated by a reducing atmosphere of hydrogen in a gas mixture of H₂/He, with molar concentration in the range from 3% to 10% of H₂, at a heating rate in the range from 1° C./min to 10° C./min and at a reduction temperature in the range from 150° C. to 350° C., for a period of time in the range from 40 to 80 min.

4. Catalytic system according to claim 3, characterized in that the activation of the catalytic system is carried out at a molar concentration in the range from 4% to 6% of H₂, at a flow rate in the range from 25 mL/min to 30 mL/min, at a heating rate in the range from 3° C./min to 8° C./min and at a reduction temperature in the range from 200° C. to 300° C., for a period of time in the range from 50 to 70 min.

5. Catalytic system according to claim 1, characterized in that the catalyst for methanol synthesis can be prepared by co-precipitation from a mixture of a solution of metal nitrates with a calcium carbonate solution, followed by calcination, with a composition that can be selected from a mixture of copper oxide and zinc oxide and a mixture of copper oxide, zinc oxide and aluminum oxide and with other added cations that can be selected from: Zr, Cr, Ga, Pd, Pt, and other metals.

6. Catalytic system according to claim 1, characterized in that the acid form of the zeolite ferrierite, with silica/alumina ratio between 60-5, is obtained by ion exchange of the sodium and potassium ions of the zeolite ferrierite for NH₄⁺ ions with a solution of salts that can be selected from ammonium nitrate, ammonium chloride and ammonium acetate, and which additionally comprises the following steps:
bring in contact, in a reflux system, stirring continuously, at a temperature of 90° C., a mass of zeolite ferrierite with a solution of ammonium salt with a concentration greater than 1 mol/L, for a time that varies in the range from 1.5 to 3 hours;
wash the zeolite paste with deionized water corresponding to four times the final volume of the mixture, after the exchange;
repeat the preceding stages of exchange and washing;
dry the acidic zeolite at a temperature in the range from 80° C. to 120° C.;
calcine the acidic zeolite at a temperature in the range from 300° C. to 700° C. for a period of time in the range from 2 to 6 hours, at a heating rate in the range from 1° C./min to 10° C./min for removal of NH₄⁺ ions.

7. Catalytic system according to claim 6, characterized in that the salt for ion exchange is ammonium nitrate; the solution of ammonium salt has a concentration in the range from 1.5 mol/L to 1.7 mol/L; the stirring time is in the range from 2 to 2.5 hours; the drying temperature is in the range from 90° C. to 100° C.; the calcining temperature is in the range from 400° C. to 500° C.; the calcining time is in the range from 4 to 5 hours; and the heating rate is in the range from 3° C./min to 8° C./min.

8. Process for direct synthesis of dimethyl ether from a synthesis gas, characterized by:
forming the catalytic system by the physical mixing of the catalyst for-methanol synthesis with the dehydration catalyst zeolite H-ferrierite;
reducing the catalytic system with H₂/He gas mixture;
on completion of reduction, starting the feed of the synthesis gas according to the following parameters: the volume ratio H₂/CO of reaction is in the range of values between 0.5 and 5; the reaction temperature varies in the range of values between 200° C. and 300° C.; the reaction pressure varies in the range of values between 1000 kPa and 10000 kPa; and the space velocity of reaction is in the range of values from 2 h⁻¹ to 100 h⁻¹.
